(19) United States
(12) Patent Application Publication
(10) Pub. No.: US 2013/0090562 A1
(43) Pub. Date: Apr. 11, 2013

(54) METHODS AND SYSTEMS FOR ASSESSING COGNITIVE FUNCTION

(71) Applicant: Jennifer RYAN, Ontario (CA)

(72) Inventor: Jennifer RYAN, Ontario (CA)

(73) Assignee: BAYCREST CENTRE FOR GERIATRIC CARE, Toronto (CA)

(21) Appl. No.: 13/646,447

(22) Filed: Oct. 5, 2012

Related U.S. Application Data
(60) Provisional application No. 61/544,763, filed on Oct. 7, 2011.

(51) Int. Cl. A61B 3/032; A61B 6/00

(52) U.S. Cl. 600/473; 600/558

(57) ABSTRACT

The present invention provides methods and systems for assessing cognitive function by comparing a subject's eye movements within and across distinct classes of images.

This application claims priority to and the benefit under 35 U.S.C. §119(e) of U.S. Provisional Patent Application No. 61/544,763 filed on Oct. 7, 2011, the disclosure of which is incorporated herein by reference in its entirety.

### FIELD OF THE INVENTION

The present invention pertains to the field of neuropsychological testing and in particular to the tracking of eye movements to assess cognitive function.

### BACKGROUND

The current standard in cognitive assessments includes a number of paper-and-pencil tasks, tasks that require motor movement, and tasks that require verbal interaction with the clinician/researcher who is conducting the assessment. If a client has motor infirmities, or cannot respond verbally (e.g., due to stroke), assessment may not be possible or the results of such testing may be inaccurate or incomplete. The current standards of neuropsychological testing are also time-consuming.

Examples of commonly used cognitive assessments are paper-based tests such as the Mini Mental Status Exam (MMSE) distributed by PAR, the Montreal Cognitive Assessment (MOCA), the Wechsler Memory Scale (WMS) distributed by Pearson, the Wechsler Adult Intelligence Scale-iv (WAIS-IV). Delis-Kaplan Executive Function System (D-KEFS), Judgment of Line Orientation, Benton Face Recognition, and Line Cancellation Tests.

Recent research has suggested that eye movement markers may be a more sensitive and precise index of cognitive functioning than standard paper-and-pencil neuropsychological assessments. Eyetracking-based neuropsychological assessment would therefore obviate the need for verbal and motor (e.g., hand) responses.

Therefore there is a need for methods and systems which apply this known correlation between cognitive function and eye movements to assess cognitive function and/or cognitive impairment in test subjects.

There is also a need for mobile monitoring systems for assessing cognitive function and/or cognitive impairment which are inexpensive and suitable for use in clinical and community settings, and which can provide an accurate assessment in a short period of time.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide methods and systems for assessing cognitive function. In accordance with an aspect of the present invention, there is provided a method of assessing cognitive function in a subject, comprising the steps of (a) presenting a plurality of images to the subject, wherein the plurality of images comprises a first subset of images and a second subset of images; (b) monitoring eye movements of the subject during presentation of the first subset of images to obtain first eye movement data; (c) monitoring eye movements of the subject during presentation of the second subset of images to obtain second eye movement data; (d) comparing the first eye movement data and the second eye movement data to determine an index of cognitive function; and (e) correlating the index of cognitive function with a degree of cognitive function in the subject, thereby assessing the cognitive function. In accordance with this aspect, the monitoring steps are carried out using an optical eyetracking system.

In accordance with another aspect of the present invention, there is provided a system for assessing cognitive function in a subject, comprising: a presentation module configured to present a first subset of images and a second subset of images to the subject; an optical eyetracking module configured to monitor the eye movement of the subject during presentation of the first subset of images and second subset of images to generate first eye movement data and second eye movement data, respectively; and a computing module communicatively linked to the optical eyetracking module and optionally the presentation module, wherein the computing module is configured to receive the first eye movement data and the second eye movement data, compare the first eye movement data and the second eye movement data to determine an index of cognitive function, and correlate the index of cognitive function with a degree of cognitive function in the subject, thereby assessing the cognitive function.

### BRIEF DESCRIPTION OF THE FIGURES

FIG. 1 is a graphical representation of data obtained from a prior art study.

FIG. 2 is a schematic representation of the Single Object Memory Task, in accordance with one embodiment of the present invention.

FIG. 3 is a graphical representation of data obtained from a prior art study.

FIG. 4 is a schematic of the Visual Paired Comparison Task, in accordance with one embodiment of the present invention

FIG. 5 is a graphical representation of data obtained from a prior art study.

FIG. 6 is a graphical representation of data obtained from a prior art study.

FIG. 7 is a schematic representation of the Spatial Association Memory Task, in accordance with one embodiment of the present invention.

FIG. 8 is a graphical representation of data obtained from a prior art study.

FIG. 9 is a schematic representation of the Object-to-Object Association Memory task, in accordance with one embodiment of the present invention.

FIG. 10 is a graphical representation of data obtained from the Single Object Memory Task.

FIG. 11 is a graphical representation of data obtained from the Visual Paired Comparison Task.

FIG. 12 is a graphical representation of data obtained from the Spatial Association Memory Task.

### DETAILED DESCRIPTION OF THE INVENTION

Unless defined otherwise, all technical and scientific terms used herein have the same meaning as commonly understood by one of ordinary skill in the art to which this invention belongs.

The present invention correlates a subject's eye movement when viewing an image with the subject's cognitive function. Examples of cognitive functions that can be assessed using the methods and systems of the present invention include, but are not limited to, long-term memory, short-term memory, working memory, language processing and comprehension, symbol processing, attention, perception, processing speed, reasoning, emotion processing, emotion recognition, executive function, and inhibition.

The present invention therefore employs eye movement markers to provide an index of cognition in an efficient manner and without requiring explicit verbal responses from the client. Eyetracking-based neuropsychological assessments are faster for the clinician/researcher to administer, allow for a wider range of clients to be tested, and provide a more precise delineation of cognitive and underlying neural integrity.

The present invention therefore provides a method for assessing cognitive function in a subject, wherein a plurality of discrete images is presented to the subject, and the subject's response (i.e., eye movements) when viewing each image is monitored using commercially available eyetracking technology. Eye movement data obtained during viewing of the images are obtained and compared, thereby providing an index of cognition. This index of cognition is correlated with an assessment in cognitive function in the subject.

In accordance with one embodiment of the present invention, the assessment of cognitive function includes an assessment, or diagnosis, of an impairment of cognitive function. In accordance with another embodiment of the present invention, the assessment of cognitive function includes an assessment, or diagnosis, of high cognitive function.

In one embodiment, the cognitive function being assessed using the method of the present invention is memory impairment. For example, for a subject with no memory impairment, the amount of viewing (e.g., the number of fixations, or the amount of time the eyes “stop” on the image) will be lower for known images or will decrease with repeated viewing of the same image. For a subject with memory impairment, it is expected that there will be little change in eye movement over the course of repeated viewing of the same image or little differentiation in eye movement between known and novel images.

Eye movements are used to reveal memory for familiar/known images, in that familiar items are typically viewed with, for example, fewer fixations or fewer distinct regions being sampled on the images than novel items, for individuals with intact memory. This correlation between eye movement and cognitive function is exploited in the present invention, and provides the basis for the presently disclosed methods and systems for assessing cognitive function.

In certain embodiments of the present invention, the methods rely on naturalistic (non-directed) viewing. In such embodiments, the invention is suitable for assessing subjects who are not capable of following instructions or communicating (for example, due to language barriers), or responding (verbally or non-verbally) to questions or instructions. Although the present invention does not require verbal or response judgment from the subject during evaluation, it is still within the scope of the present invention to incorporate a verbal or response judgment component to the cognitive function assessments.

### Methods of Assessing Cognitive Function

The methods of the present invention rely on the collection of data relating to the subject's eye movement when viewing an image. Eye movement data can be compiled and analyzed in several different ways. A selection of commonly used characterizations of viewing are defined as follows. This list is representative, and is not intended to be limiting.
Number of fixations: the number of discrete pauses of the eyes for a display.
Fixation duration: the length of time in which the eye pauses on a display, wherein median or mean fixation duration to a display are calculated.
Number of regions fixated: the number of discrete regions sampled within a display.
Location of fixations: the location in the image on which the eye is fixated.
Spatial distribution of fixations: the total area explored by the eyes on the image.
Temporal order of fixations
Measurement of saccades, including parameters such as amplitude, acceleration, velocity and duration.
Constraint/entropy with the spatial and temporal distributions of fixations: how the location or duration of the current fixation may predict the location or the duration of the next fixation.
Comparison of the similarity of eye movement patterns across images, in spatial and/or temporal distribution.
Characteristics of eye fixations (e.g., duration, location, number, order) with respect to particular regions of interest in an image.
The number of transitions that are made by the eyes between pre-specified regions of interest.
Smooth pursuit movements, including parameters such as acceleration, velocity and duration.

The present invention also provides for the monitoring of pupil dilation as a measure of cognitive function as appropriate.

Sampling of visual materials can be characterized in terms of overall viewing at the level of an entire experimental display, or directed viewing at the level of regions, objects, or stimuli within that display.

During the course of an assessment task, a series of images is presented to the subject. In accordance with the present invention, each image is presented for a predetermined period of time, the duration of which is determined according to the assessment task being conducted and the type of eye movement data being sought. For example, an image may be presented for a shorter duration, such as, but not limited to, 100 milliseconds; an image may also be shown for a longer duration, such as, but not limited to, 5 seconds.

In accordance with the present invention, a plurality of images is presented to the subject during the course of an assessment task, wherein the plurality of images comprises a first subset of images and a second subset of images.

In one embodiment, the first subset of images is a single image not previously viewed by the subject, and the second subset of images is a single image previously viewed by the subject. In one embodiment, the first and second subsets of images are presented simultaneously. In another embodiment, the first and second subsets of images are presented sequentially.

In one embodiment, the first subset of images consists of images not previously viewed by the subject, and the second subset of images consists of images previously viewed by the subject.

In one embodiment, the first subset of images comprises a first image, wherein the first image has not been previously viewed by the subject, and the second subset of images comprises a repeated presentation of the first image. In this embodiment, eye movement data is obtained by monitoring the eye movements of the subject during the presentation of the first image as well as during each of the subsequent presentations of the first image.

In one embodiment, the first subset of images and second subset of images each consist of images depicting a plurality of items in a defined spatial arrangement, wherein the first and second subsets differ only in the relative spatial arrangement of the plurality of items.

In some embodiments of the present invention, a fixation screen is presented for a defined period of time between each test image. The duration of the fixation screen can be adjusted to test the range of conditions under which intact versus impaired cognitive function is observed.

The number of repetitions in a given test is that which is sufficient to provide an index of cognitive function. Determination of the number of repetitions is made with consideration of factors including, but not limited to, the length of duration of an individual exposure, the overall number of images presented in a given test, and/or how distinct each image is from other images presented in the test. Accordingly, the recitation of a number of repetitions in the description of any tasks disclosed herein is not intended to be limiting, and it is understood that any number of repetitions as is determined by a worker skilled in the relevant art to be sufficient to provide an index of cognitive function falls within the scope of the present invention.

The duration of each task is variable, and depends on, for example, the number of images presented, the duration of the familiarization phase for each image, and the amount of repetition for each image. Where a fixation screen is used between presentation of each image in the familiarization and test phases, the duration of the fixation screen will also impact the overall duration of the task. Where a delay between the familiarization phase of each image and the subsequent test display of either the same or altered image, is employed, the overall duration of the task is impacted.

### Tasks for Assessment of Cognitive Function

The methods of the present invention can be carried out using a variety of different tasks to assess cognitive function. Non-limiting examples of such tasks are set out below.

Single Object Memory Task (Known/Familiar vs. Unknown/Novel)

The task is designed to examine visual memory for single objects, which is thought to rely on visual cortical areas and regions of the medial temporal lobe, in particular, the perirhinal cortex.

In this task, the subject is presented with a series of images of distinct items (e.g., faces, objects, abstract non-nameable images), wherein each image is presented for a predetermined length of time. The series of images includes a combination of novel (unknown or not previously viewed) and known (familiar or previously viewed) images. Novel images are presented only one time during the course of the task. Presentation of the items will be randomized. Known images can include images familiar to the subject or are otherwise known from the subject's previous experience. The known images can also include images that are presented repeatedly over the course of the test. This repeated presentation of an image is referred to as a familiarization phase.

The length of time for viewing the images during this familiarization phase can be adjusted to test the range under which subsequent eye movement memory effects (as described below) are observed. A subset of the presented items are shown once only (novel), other items are each shown multiple times (repeated). In one embodiment, there is a fixation screen in between presentation of each item.

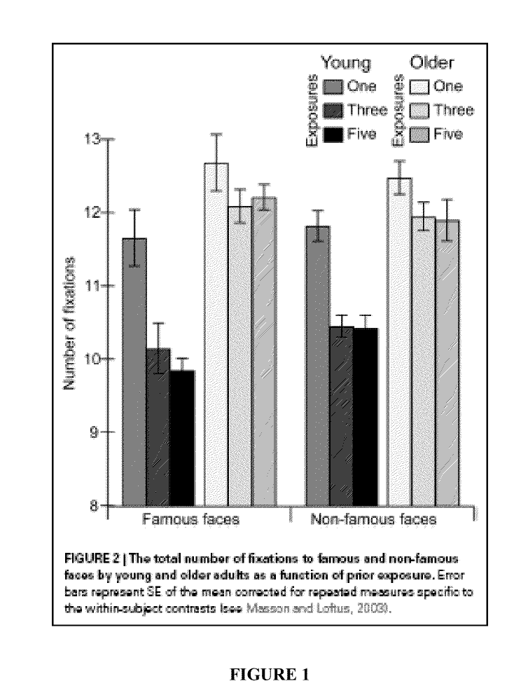

The decrease in viewing behavior that accompanies increased exposure was demonstrated in Heisz, J. J. & Ryan, J. D. (2011) (The effects of prior exposure on face processing in younger and older adults. Frontiers in Aging Neuroscience, 3:15. doi: 10.3389/fnagi.2011.00015). This work provided the foundational research for the design of the Single Object Memory Task. Representative data obtained in this study is presented in FIG. 1.

In the Single Object Memory Task, the subject's eye movements are monitored during the viewing of each image. It is expected that, for a subject with no memory impairment, the amount of viewing (for example, but not limited to, the number of fixations, or the amount of time the eyes “stop” on the image) will decrease across repetitions. Also, the spatial distribution of the eye movements across the image should decrease (i.e., the total area explored by the eyes on the image).

This task can be adapted to test higher memory function/achievement, by making the memory test more difficult by including a subset of novel and repeated images that are very similar to each other, thereby making it more difficult to form separate memories of each and distinguish novel from repeated.

A measure of change between novel and repeated ((novel-repeated)/novel) or across repetitions ((1^{st} presentation−nth presentation)/1^{st} presentation) is generated for each eye movement measure (e.g., number and/or duration of fixations). In this way, an index of cognitive function is generated. A score of 0 (or below) indicates that there is no memory that has been maintained for the repeated objects (i.e., viewing of repeated items is similar to viewing of novel items). A score higher than 0 indicates that the subject has memory for those items that are repeated.

Examining the change in eye movements across repetition levels (e.g., 1 exposure, 3 exposures), or familiarization duration (e.g., 1 second each viewing, 5 seconds each viewing), indicates how fast memories are being formed. The slope of the change in eye movements across levels captures the rate of learning (a slope of 0 indicates no learning, a positive slope indicates learning).

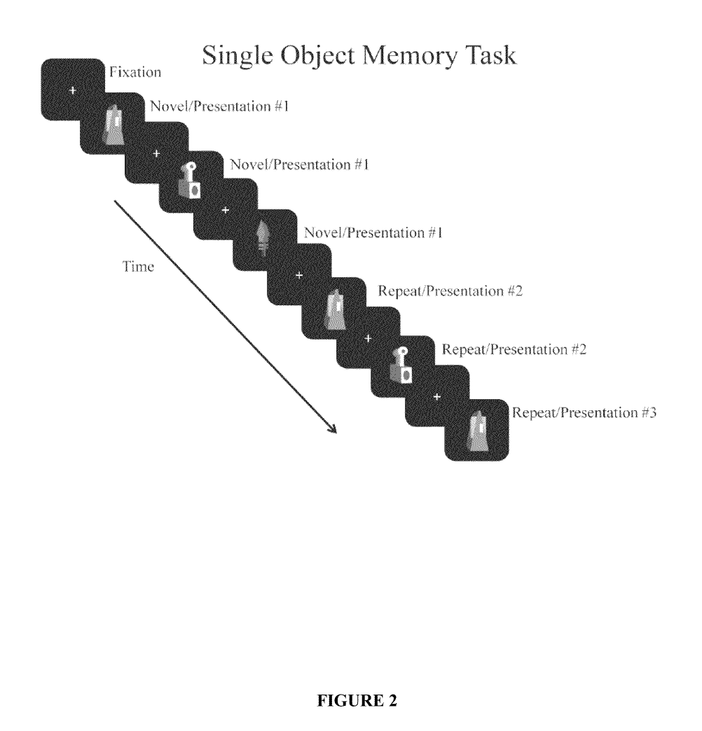

A schematic representation of one embodiment of the Single Object Memory Task is shown in FIG. 2. In this embodiment, single images are shown, one at a time, with a fixation screen in between each image. Some images are shown only once (Novel/Presentation #1), while some images are shown multiple times (Repeat/Presentation #2, etc.). The duration of image presentation, as well as the delay in between presentations of the same image, can be varied. Such manipulations, as well as similarity of the images, can be used to increase the difficulty of the task. Memory (cognitive function) is indexed through a decrease in viewing behavior (e.g., number of fixations) to the Repeat images compared to the Novel/Presentation #1 images.

### Visual Paired Comparison Task

This task is designed to examine visual memory for single objects across varying delays, which is thought to rely on visual cortical areas, and the medial temporal lobe, particularly as the delay increases.

In the visual paired comparison task, two images are presented simultaneously, one image being a known (previously viewed/repeated) image, and the other being a novel image.

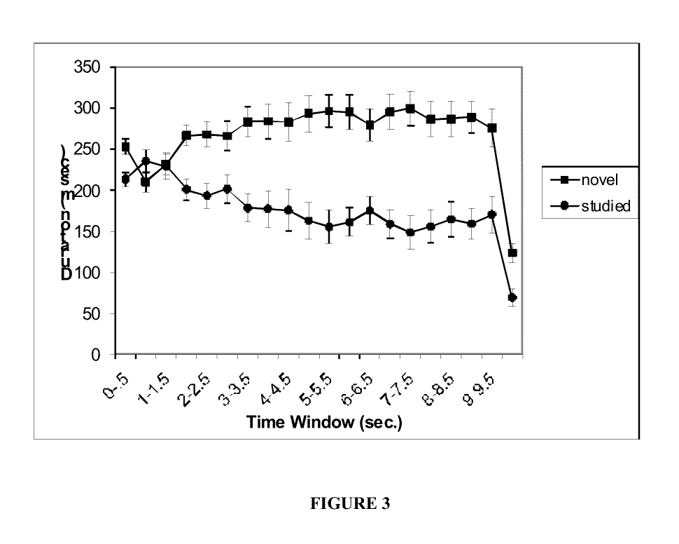

An increase in viewing towards a novel image, when a novel and previously viewed image (studied) are presented simultaneously, was demonstrated in Ryan, J. D., Hannula, D. E., & Cohen, N. J. (2007) (The obligatory effects of memory on eye movements. Memory, 15(5), 508-525). This work provided the foundational research for the design of the Visual Paired Comparison (Preferential Viewing) Task. Representative data obtained in this study is presented in FIG. 3.

Again, the known image can include images familiar to the subject or are otherwise known from the subject's previous experience. The known images can also include images that are presented repeatedly during a familiarization phase.

During such a familiarization phase, the subject is shown a series of images of distinct items (e.g., faces, objects, abstract non-nameable images), one at a time, for a predetermined period of time. Items are repeated multiple times. In one embodiment, there is a fixation screen in between presentation of each item. The amount of repetition of each item, the period of viewing time and the duration of fixation screen are each independently adjustable to test the range under which subsequent eye movement memory effects are observed.

In one embodiment, a delay is imposed after the familiarization phase and the beginning of the test phase. This delay between viewing of a stimulus in the familiarization phase and viewing of the same stimulus in the test phase is adjustable for each item to examine immediate versus longer-term memory. Subjects are shown pairs of items, one item on each side of the screen, for a pre-determined amount of time. These pairs of items consist of one previously viewed (repeated) image, and one novel image. In one embodiment, there is a fixation screen in between each presentation of a pair of items. Presentation of the items is pseudo-randomized to capture a range of delay conditions, and examine memory performance over time.

This task can be adapted to test higher memory function/achievement by varying how similar the novel and known images are. The more similar two images are, the more difficult it should be to distinguish between the two, but if a subject has superior memory, their eye movements will indicate that they are able to distinguish between very similar images.

The subject's eye movements are monitored during the viewing of each pair of images. It is expected that a subject with no memory impairment should direct more eye movements (e.g., duration of viewing, fixations) to the novel item when the pairs of items (novel+repeated) are presented. A preferential viewing memory score can be calculated as: ((Duration of Viewing to Novel−Duration of Viewing to Repeat)/Duration of Viewing to Novel) A score of 0 (or below) indicates that there is no memory that has been maintained for the repeated objects (i.e., viewing of novel items is equal to viewing of repeated items). A score higher than 0 indicates that the subject has memory for those items that are repeated.

A slope can be generated for the ratios across the delays to determine the stability of memory over time (here, a slope of 0 indicates no change in memory over time, a negative slope indicates forgetting over longer delays, a positive slope indicates impaired shorter-term memory processes relative to longer-term memory processes).

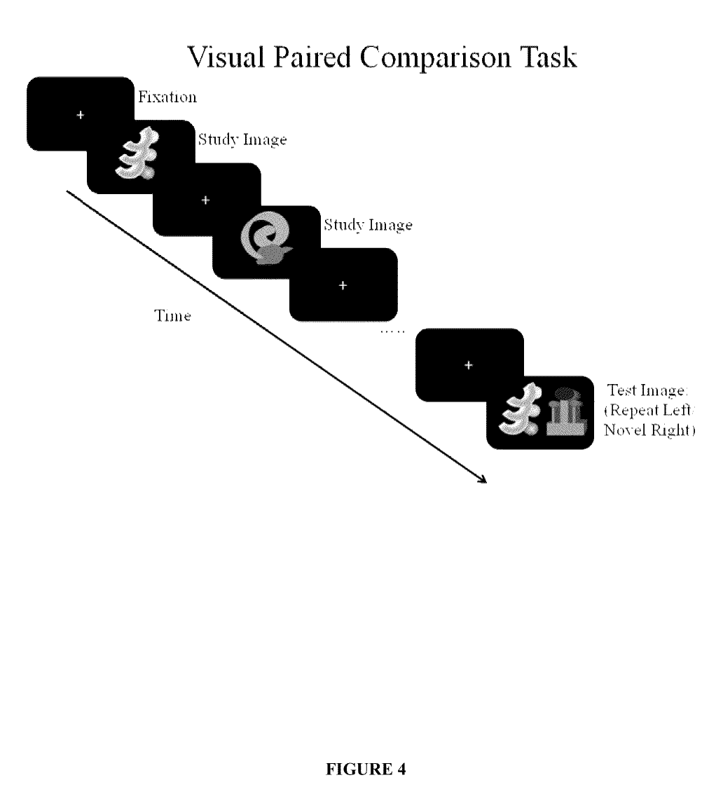

A schematic representation of one embodiment of the Visual Paired Comparison Task (also known as the Preferential Viewing Task) is shown in FIG. 4. In this embodiment, study images are shown, one at a time, with a fixation screen in between each image. Test images contain one Novel image (never previously viewed) and one Repeat image (a previously viewed study image); left/right presentation of the Novel and Repeat images varies across test trials. The duration of image presentation can be varied, as well as the delay in between presentations of the Study and Test images. Such manipulations, as well as similarity of the images, can be used to increase the difficulty of the task. Memory (cognitive function) is indexed by greater viewing (e.g., greater duration of viewing) to the Novel image compared to the Repeat Image. In this way, an index of cognitive function is generated.

### Memory for Spatial Associations

The task is designed to examine visual memory for the spatial relationships among objects, which is thought to rely on the prefrontal cortex (at short delays), and medial temporal lobe (at short and longer delays)

In this task, the subject is presented with a series of images in a familiarization phase, wherein each image is presented for a predetermined length of time, and the length of time for viewing the images during this familiarization phase can be adjusted to test the range under which subsequent eye movement memory effects are observed. Each of the images comprises a plurality of objects (including, but not limited to, known objects, abstract non-nameable objects, faces) in a defined spatial arrangement. Examples include images of everyday scenes (e.g., an arrangement of furniture in a living room), or an assembly of everyday objects in a defined spatial arrangement.

Following the familiarization phase, a delay is imposed prior to the beginning of the test phase. Images in the test phase consist of those that are re-presented in the same exact format (repeated images), and images that have been seen in the familiarization phase, but in the test phase have been altered (altered images). This alteration can be in the form of a change in the spatial arrangement of the objects, or in the removal or addition of an object from the image. The delay between viewing of a stimulus in the familiarization phase and viewing of the same or altered stimulus in the test phase is adjustable for each item to examine immediate versus longer-term memory. In one embodiment, there is a fixation screen in between each presentation of a pair of items. Presentation of the items is pseudo-randomized to capture a range of delay conditions, and examine memory performance over time.

This task can be adapted to test higher memory function/achievement, by making the memory test more difficult by including a subset of altered and repeated images that are very similar to each other, thereby making it more difficult to form separate memories of each and distinguish altered from repeated.

The subject's eye movements are monitored during the viewing of each image. It is expected that a subject with no memory impairment should distinguish between repeated images and altered images. Specifically, viewing should be preferentially directed (e.g., number and/or duration of fixations) to the location of the image that has undergone a change in the altered images compared to a similar region of the repeated images.

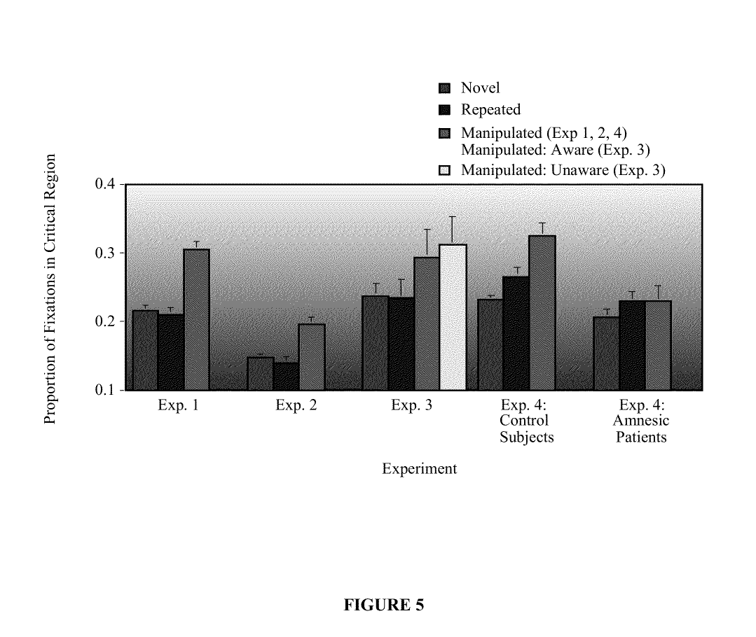

An increase in viewing towards the critical regions of a scene that have been altered (manipulated) from a prior viewing, compared to critical regions from the same scenes when viewed as either a novel, or repeated image was demonstrated in Ryan, J. D., Althoff, R. R, Whitlow, S., Cohen, N J. (2000) (Amnesia is a deficit in relational memory. Psychological Science, 11, 454-461). Representative data obtained in this study are presented in FIG. 5.

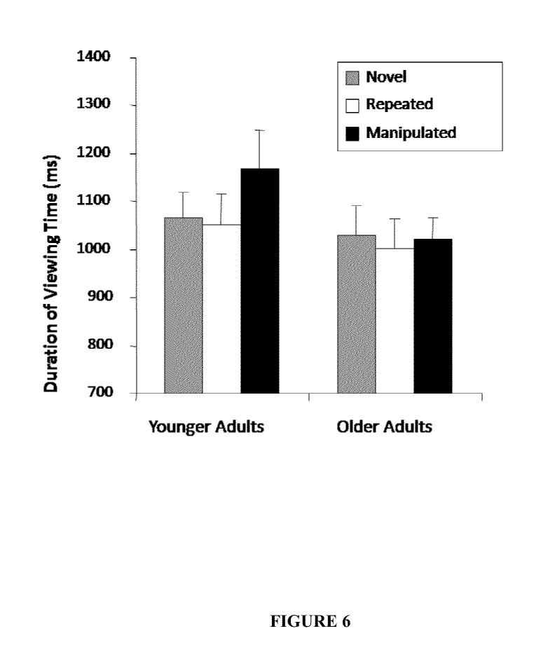

This effect was also demonstrated in Ryan, J. D., Leung, G. L., Turk-Browne, N. B., & Hasher, L. (2007) (Assessment of age-related changes in inhibition and relational memory binding using eye movement monitoring. Psychology and Aging, 22, 239-250), which showed that such effects are evident for younger adults, but are absent in older adults. Representative data obtained in this study are presented in FIG. 6.

The work described in these references provided the foundational research for the design of the Spatial Association Memory Task.

A ratio (((Altered presentation−1st presentation)/(1^{st} presentation))−((Repeated presentation−1st presentation)/1^{st} presentation))) is generated for each eye movement measure for each delay condition. A score of 0 (or below) indicates that there is no memory that has been maintained for the spatial arrangements of the objects (i.e., viewing of altered scenes is similar to viewing of repeated scenes). A score higher than 0 indicates that viewers have memory for the spatial arrangements of the objects within the scenes.

Examining the slope of the ratios across delay conditions levels (immediate, short, long) indicates how fast memories are formed/forgotten (a slope of 0 indicates no difference between shorter-term and longer-term memory processes, a negative slope indicates forgetting over longer delays, a positive slope indicates impaired shorter-term memory processes relative to longer-term memory processes).

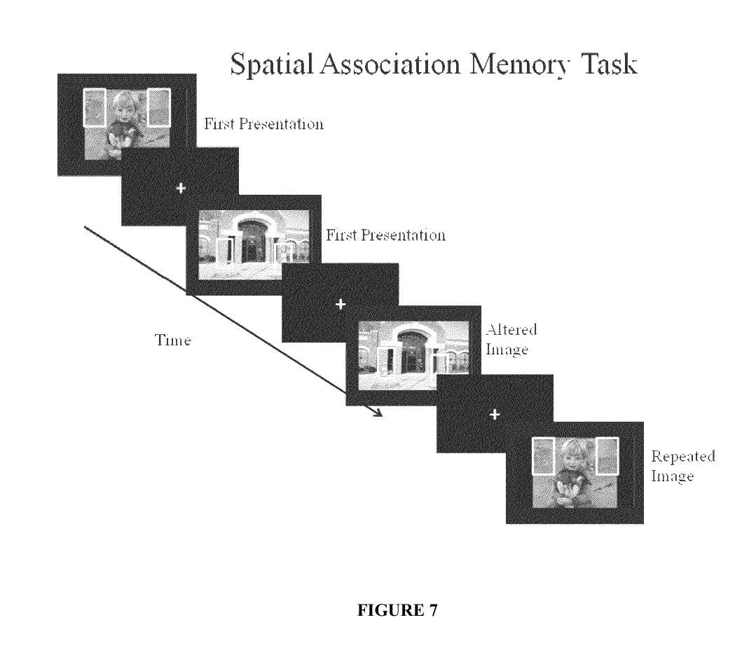

A schematic representation of one embodiment of the Spatial Association Memory task is shown in FIG. 7. In this embodiment, images are shown, one at a time, with a fixation screen in between each image. Images are initially presented in an original form (First Presentation) and are subsequently presented as the exact same image (Repeated Image) or contain a change (Altered Image). In the example above, the newspaper dispenser has moved from the right side of the image to the left, as noted by the boxed outlines. Note that the boxes in each image denote the critical regions on the image where a change may occur. The boxes are for illustrative purposes only, and are not presented to the viewer. The duration of image presentation can be varied, as well as the delay in between presentations of the First Presentation and the Altered or Repeated Image. Such manipulations, as well as similarity of the images, can be used to increase the difficulty of the task. Memory is indexed by an increase in viewing to the critical regions of change in Altered Images relative to the First Presentation, baselined by any change in viewing to the critical regions (in which no change has occurred) for the Repeated Images relative to their respective First Presentations. In this way, an index of cognitive function is generated.

### Object-to-Object Association Task

This task is designed to examine visual memory for the relationships among objects, which is thought to rely on the prefrontal cortex (at short delays), and medial temporal lobe (at short and longer delays)

During a familiarization phase, two images (e.g., pictures of real objects, abstract figures, real-world scenes) may be presented simultaneously or one image may be presented, followed by an overlay of a second image on the first, thereby allowing the presentation of each image to be associated with the other. In one embodiment, presentation of the associated images is repeated multiple times. In one embodiment, there is a fixation screen in between presentation of each associated presentation. The amount of repetition of each item, the period of viewing time and the duration of fixation screen are each independently adjustable to test the range under which subsequent eye movement memory effects are observed.

In one embodiment, a delay is imposed after the familiarization phase and the beginning of the test phase. This delay between viewing of the stimuli in the familiarization phase and viewing of the same stimulus in the test phase is adjustable for each item to examine immediate versus longer-term memory. Subjects are shown one image of a previously studied associated pair, for a pre-determined amount of time, immediately followed by two images presented simultaneously, one of which is the associated member of the previously presented image (‘match’ image) and the other which had not been paired with the previously presented image (‘nonmatch’ image). Presentation of the items is pseudo-randomized to capture a range of delay conditions, and examine memory performance over time.

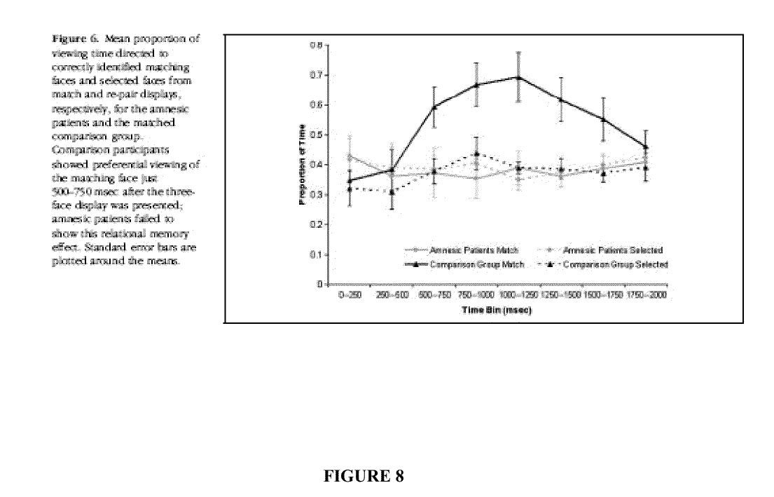

An increase in viewing towards the image that “matched” (had been previously associated with) a presented background image was demonstrated in Hannula, D. E., Ryan, J. D., Tranel, D. & Cohen, N. J. (2007) (Rapid onset relational memory effects are evident in eye movement behavior, but not in hippocampal amnesia. Journal of Cognitive Neuroscience, 19, 1690-1705). Such effects are absent in amnesic patients who have damage to the medial temporal lobe including the hippocampus; representative data obtained in this study are present in FIG. 8. This work provided the foundational research for the design of the Object-to-Object Association Memory Task.

This task can be adapted to test higher memory function/achievement by varying how similar the matching and nonmatching images are. The more similar two images are, the more difficult it should be to distinguish between the two, but if a subject has superior memory, their eye movements will indicate that they are able to distinguish between very similar images.

The subject's eye movements are monitored during the viewing of the match/nonmatch pair of images. It is expected that a subject with no memory impairment should direct more eye movements (e.g., duration of viewing, fixations) to the image that had been previously associated with the preceding image when the match and nonmatch images are presented simultaneously. A ratio of match:nonmatch is generated for each eye movement measure. A score of 1 (or below) indicates that there is no memory that has been maintained for the associated pair (i.e., viewing of matching items is equal to viewing of nonmatching items). A score higher than 1 indicates that the subject has memory for the associated pairs.

A slope can be generated for the ratios across the delays to determine the stability of memory over time (here, a slope of 0 indicates no change in memory over time, a negative slope indicates forgetting over longer delays, a positive slope indicates impaired shorter-term memory processes relative to longer-term memory processes).

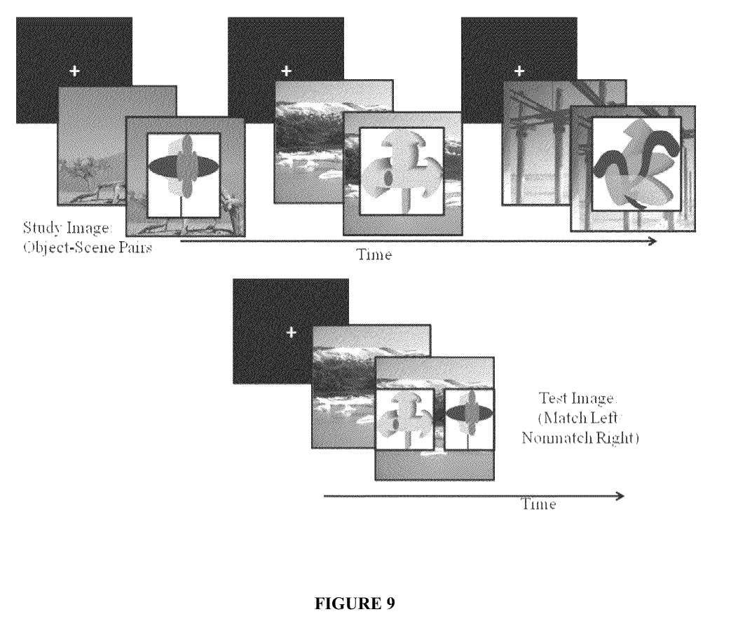

A schematic representation of one embodiment of the Object-to-Object Association Task is shown in FIG. 9. In this embodiment, following a fixation image, a Study Image is presented in which a background scene is first presented alone, followed by an overlay of an object onto the scene. This sequence allows the viewer to form Object-Scene associations in memory. Following the presentation of the Study Images, the Test Images are presented in which a background scene is first presented alone, followed by an overlay of two objects onto the scene. Both objects have been previously viewed during the study phase, however only one has been previously associated with the background scene (Match Image, as depicted on the left). The other image was not previously associated with the background scene (Nonmatch Image). The duration of image presentations can be varied, as well as the delay in between presentations of the Study Image and its respective Test Image. Such manipulations, as well as similarity of the images, can be used to increase the difficulty of the task. Memory (cognitive function) is indexed through an increase of viewing to the Object Image that had been previously associated with the background scene (Match Image). In this way, an index of cognitive function is generated.

### Emotion Processing Task

This task is designed to examine a subject's ability to process and distinguish emotions, which is typically disrupted in disorders such as autism spectrum disorders, in people who have lesions to the amygdala, and in people who have depression.

In this task, a subject is shown a series of distinct faces expressing different emotions (including but not limited to, neutral, anger, disgust, fear, happiness), one at a time. Some faces may be presented in isolation, whereas others may be presented within a context that is either congruent or incongruent with the emotion that is expressed by the face (e.g., a face displaying disgust is presented with a body conveying the emotion of anger) The length of time for viewing the images during this familiarization phase can be adjusted to test the range under which successful emotion processing can be observed. Presentation of the items is randomized. In one embodiment, there is a fixation screen in between presentation of each item.

It is expected that, in subjects whose ability to process and distinguish emotions is intact, the distribution of viewing across (e.g., number and/or duration of fixations) the face features should distinguish between the different emotions, and such viewing should be impacted by the context onto which the face is presented.

Ratios that contrast across the different emotion conditions are generated for each eye movement measure of viewing to the face and/or face features (e.g. anger:disgust). A score of 1 (or below) indicates that there the viewer does not distinguish between the emotions presented, whereas a change from 1 indicates an ability to process and distinguish between emotions.

### Categorization/Symbol Processing/Language Comprehension Task

This task is designed to examine the ability of the subject to process spoken and/or written language, evaluate and categorize objects. Deficits in these abilities may be present in disorders including, but not limited to, dementia and aphasia.

In this task, a subject is shown a series of images that depict one or more objects and/or people. In one embodiment, the images involve the objects and/or people interacting or otherwise engaging in a particular action (e.g., one person pushing another). In another embodiment, the objects and/or people are presented without any such engagement. For example, in such an embodiment, four circles of different shapes and sizes are presented simultaneously in different spatial locations on the screen, or four different types of dogs and one cat are presented simultaneously in different spatial locations on the screen. Objects presented within an image may be from the same basic, superordinate or subordinate categories, or one or more objects may be from a different basic, superordinate or subordinate category as the other objects. In one embodiment, a subset of the images is presented in isolation, whereas other images are accompanied by either a spoken (auditory presentation) or written sentence. The length of time provided for viewing the images depends on the condition (presented in isolation, presented with spoken sentence, presented with written sentence). In one embodiment, there is a fixation screen in between presentation of each item. Images (presented either with or without their corresponding auditory or visual sentences) may be repeated or may be presented only once.

This task can be adapted to test higher cognitive functioning, by making the spoken and written word sentences more difficult (i.e., use a higher level of vocabulary). Monitoring eye movements while viewing the images during this modified task can reveal comprehension of the sentence. This task can also be modified to test higher cognitive functioning by increasing the similarity of the items within the symbol processing test.

It is expected that the distribution of viewing across the items should be tied to the accurate categorization of the items (e.g., more viewing to the item that does not belong to the same category). Viewing order and preference to the items within the image should correspond to the comprehension of the spoken/written language. For instance, the order by which the viewer fixates the items should correspond to the active/passive nature of the sentence (e.g., “the boy pushed the girl” should elicit viewing first to the boy, then to the girl; whereas “the girl was pushed by the boy” should elicit viewing first to the girl and then to the boy).

Ratios will contrast the distribution of viewing across objects. For example, for trials in which one object is presented amongst other objects from a different basic, superordinate or subordinate category, it is expected that viewing (e.g., number and/or duration of fixations) will be preferentially directed towards the oddball object compared to the average of the other objects if the viewer has comprehension regarding the semantic categorization of objects (ratio greater than 1). In the embodiment in which the images are presented with a spoken or written sentence, a score from 0-1 is also derived that indicates the preferential order of viewing and the extent to which the order of viewing matched the order of words as presented in auditory or visual sentence. A score of 1 indicates perfect concordance of viewing with the sentence presentation, and therefore intact symbol processing and language comprehension, whereas a score of 0 means no concordance between viewing with the sentence presentation, poor symbol processing and language comprehension.

### Assessment of Visuo-Spatial Perception Task

This task is designed to examine visual attention/inattention to areas of space within the field of view.

In this task, a subject is shown images that depict one or more objects and/or people. Images are repeated, and flipped in left-right orientation. A fixation screen is presented in between each of the images.

It is expected that, across the collection of images, the distribution of viewing should be balanced across the left and right sides of the images, with reference to the number of objects contained within each side. The extent to which the viewer exhibits visual neglect is evidenced by more viewing to one side versus another.

Ratios will contrast left:right distribution of viewing. A ratio at or close to 1 indicates little to no visual inattention; ratios that are skewed greater than 1, or less than 1 indicate the presence of visual inattention, and the side on which the neglect is occurring.

### Systems for Assessing Cognitive Function

The present invention demonstrates the effectiveness of using eye movements to assess cognitive function by monitoring a subject's eye movement while viewing a series of images as described, for example, in the tasks described above. Accordingly, the present invention also provides a system for assessing cognitive function in a subject, comprising a presentation module configured to present a first subset of images and a second subset of images to the subject, and an optical eyetracking module configured to monitor the eye movement of the subject during presentation of the first and second subsets of images to generate first and second eye movement data. In accordance with the present invention, the system further comprises a computing module communicatively linked to the optical eyetracking module, wherein the computing module is configured to receive the first and second eye movement data, and compare them to determine an index of cognitive function. This index of cognitive function is correlated with a degree of cognitive function in the subject, thereby assessing the cognitive function.

Different computer platforms are suitable for use in the presently disclosed system, including, but not limited to, home computers, laptop computers, PDAs or any mobile computer platform.

Particularly preferred are computer platforms that are readily transportable and/or mobile, such as laptops, smartphones and computer tablets.

In one embodiment of the present system, the presentation module comprises means for displaying an image, including but not limited to a computer monitor or the screen of a laptop or handheld computing device. In one embodiment, the presentation module comprises any means for projecting images onto a screen or other suitable surface, or means for transmitting images for display on, for example, a television screen.

In one embodiment of the present system, the eyetracking module comprises a means for tracking the eye movement of the subject while viewing images.

A number of different eyetracking technologies, including optical, electrical or magnetic based methods, are known in the art and are available and suitable for monitoring eye movement in accordance with the present invention. Particularly suitable for use with the present invention are optical eyetracking technologies, being typically non-invasive and relatively inexpensive. In one embodiment, the eyetracking module employs infrared (IR)-based technology. Moreover, there are a number of commercially available, inexpensive, IR-based technologies that can be readily adapted for use with mobile computing platforms to provide mobile systems for assessing cognitive function.

In one embodiment, the present assessment system comprises a commercially available infrared eyetracker system. Such systems include, but are not limited to, those manufactured by Mirametrix (Westmount, QC, Canada), SensoMotoric Instruments (SMI, Berlin, Germany), Applied Science Laboratories (Bedford, Mass.), and SR Research (Kanata, ON, Canada).

In one embodiment, the present invention is implemented using IR LEDs to illuminate the eyes.

In one embodiment, eye movements are monitored using a native webcam attached to a home computer or laptop.

In one embodiment, the present invention incorporates a remote, IR camera-based eyetracking system.

The present assessment system comprises a computing module for receiving eye movement data from the eye tracking module, and comparing the data to determine an index of cognitive function. This index of cognitive function is correlated with a degree of cognitive function in the subject, thereby assessing the cognitive function. In one embodiment, the computing module is also communicatively linked to the presentation module.

It will be appreciated that, although specific embodiments of the invention have been described herein for purposes of illustration, various modifications may be made without departing from the spirit and scope of the invention. In particular, it is within the scope of the invention to provide a computer program product or program element, or a program storage or memory device such as a solid or fluid transmission medium, magnetic or optical wire, tape or disc, or the like, for storing signals readable by a machine, for controlling the operation of a computer according to the method of the invention and/or to structure some or all of its components in accordance with the system of the invention.

Acts associated with the method described herein can be implemented as coded instructions in a computer program product. In other words, the computer program product is a computer-readable medium upon which software code is recorded to execute the method when the computer program product is loaded into memory and executed on the microprocessor of the wireless communication device.

Acts associated with the method described herein can be implemented as coded instructions in plural computer program products. For example, a first portion of the method may be performed using one computing device, and a second portion of the method may be performed using another computing device, server, or the like. In this case, each computer program product is a computer-readable medium upon which software code is recorded to execute appropriate portions of the method when a computer program product is loaded into memory and executed on the microprocessor of a computing device.

Further, each step of the method may be executed on any computing device, such as a personal computer, server, PDA, or the like and pursuant to one or more, or a part of one or more, program elements, modules or objects generated from any programming language, such as C++, Java, PL/1, or the like. In addition, each step, or a file or object or the like implementing each said step, may be executed by special purpose hardware or a circuit module designed for that purpose.

The invention will now be described with reference to specific examples. It will be understood that the following examples are intended to describe embodiments of the invention and are not intended to limit the invention in any way.

### EXAMPLES

### Example 1

### Task: Memory for Single Objects

Rationale: examine visual memory for single objects.

Procedure: Subjects are shown 10 distinct items (faces and/or objects), one at a time, for 3 sec. each. Five of the items are shown once only (novel), the other 5 items are each shown 5 times (repeated). Presentation of the items is randomized. There is a 1-second fixation screen in between presentation of each item.

Duration of Task: 2 minutes.

Scoring: A measure of change of viewing for novel versus repeated images across time is generated for each eye movement measure (e.g., number and/or duration of fixations). A score of 0 (or below) indicates that there is no memory that has been maintained for the repeated objects (i.e., viewing of repeated items is similar to viewing of novel items). A score higher than 0 indicates that the subject has memory for those items that are repeated. Examining the ratios across repetition levels (1-5) indicates how fast memories are being formed. Additionally, a slope of the ratios across levels captures the rate of learning (a slope of 0 indicates no learning, a positive slope indicates learning).

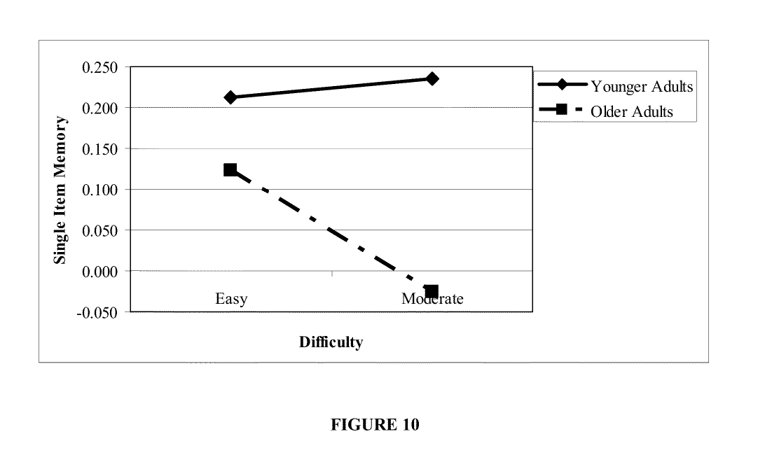

FIG. 10 is a graphical summary of data obtained from 37 younger adults and 14 older adults from the Single Object Memory task, in which the similarity of the presented images varied between the difficulty levels. The objects in the Moderate condition were visually more similar to each other (e.g., same color, similar shape) than the objects in the Easy Condition. The findings show a decline in memory for older adults relative to younger adults, in particular for the Moderate difficulty level. The single item memory score was calculated as the change in the number of fixations across presentations of repeated images baselined for the change in fixations across the presentation of the novel images (i.e., correcting for change in viewing behavior across time):

((Number of Fixations Presentation #1-Number of Fixations Presentation #N)/Number of Fixations Presentation #1)−((Number of Fixations Novel Image#First-Number of Fixations Novel Image #Last)/Number of Fixations Novel Image#Last)

### Example 2

### Task: Visual Paired Comparison Task (also called Preferential Viewing Task)

Rationale: examine visual memory for single objects across varying delays.

Procedure: Subjects are shown 10 distinct items (faces and/or objects), one at a time, for 3 sec. each. All 10 items are repeated 5 times. There is a 1-second fixation screen in between each item. Following a delay (immediate test or 2 minutes), viewers are shown 10 pairs of items, one item on each side of the screen, for 3 sec. each. These pairs of items consist of one previously viewed (repeated) image, and one novel image. There is a 1-second fixation screen in between each presentation of a pair of items. Presentation of the items is pseudo-randomized so that all of the 2-minute delay items are studied first, followed by the immediate study items, then the test pairs for the immediate condition, and finally, the test pairs for the 2-minute delay condition.

Duration of Task: 4 minutes.

Scoring: A score comparing viewing to the novel image versus viewing to the repeated image is generated for the time that is spent looking at each item. A score of 0 (or below) indicates that there is no memory that has been maintained for the repeated objects (i.e., viewing of novel items is equal to viewing of repeated items). A score higher than 0 indicates that viewers have memory for those items that are repeated. A slope is generated for the ratios across the two delays to determine the stability of memory over time (here, a slope of 0 indicates no change in memory over time, a negative slope indicates forgetting over longer delays, a positive slope indicates impaired shorter-term memory processes relative to longer-term memory processes).

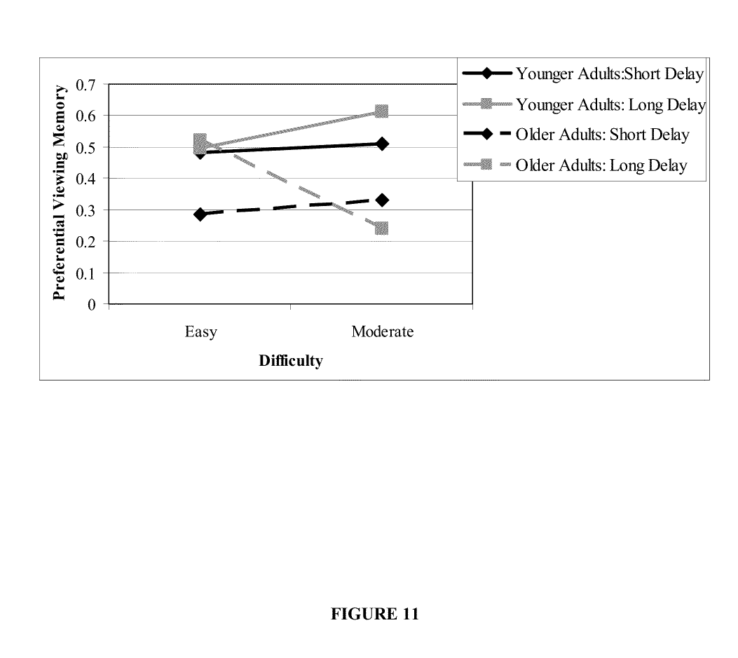

FIG. 11 is a graphical summary of data obtained from 42 younger adults and 16 older adults from the Visual Paired Comparison (Preferential Viewing) Memory task, in which the similarity of the presented images varied between the difficulty levels. The objects in the Moderate condition were visually more similar to each other (e.g., same color, similar shape) than the objects in the Easy Condition, and the delay between study and test images was either short (approximately 20 seconds) or long (approximately 2 minutes). Older adults show memory declines relative to younger adults when they are tested after a short delay on the Easy condition, and after a short and long delay for the Moderate condition. The preferential viewing memory score was calculated as ((Duration of Viewing to Novel−Duration of Viewing to Repeat)/Duration of Viewing to Novel).

### Example 3

### Task: Memory for Spatial Associations

Rationale: examine visual memory for the spatial relationships among objects.

Procedure: Subjects are shown a series of everyday scenes (e.g., an arrangement of furniture in a living room) one at a time, for 3 sec. each. There are 18 scenes in total, and each scene is shown twice. In the immediate delay condition, the second presentation of the scene immediately follows the first presentation of the scene. In the short delay condition, the second presentation of the scene occurs 8 seconds following the presentation of the first scene (two intervening scenes, an immediate trial, will occur). In the long delay condition, the second presentation of the scene occurs 36 seconds following the first presentation (intervening scenes will include two immediate trials, two short trials, and the first presentation of a scene from a long delay trial). Three of the scenes in each delay condition are re-presented in the same exact format (repeated), three of the scenes in each delay condition contain a change in the spatial arrangement of the objects within the scene (altered). Presentation of the scenes is pseudo-randomized in order to accommodate the delay conditions. There is a 1-second fixation screen in between the presentation of each scene.

Duration of Task 2.5 minutes.

Scoring: A score contrasting viewing of altered images versus viewing of repeated images relative to their respective, baseline, initial presentations is generated for each eye movement measure for each delay condition. A score of 0 (or below) indicates that there is no memory that has been maintained for the spatial arrangements of the objects (i.e., viewing of altered scenes is similar to viewing of repeated scenes). A score higher than 0 indicates that viewers have memory for the spatial arrangements of the objects within the scenes. Examining the slope of the ratios across delay conditions levels (immediate, short, long) indicates how fast memories are formed/forgotten (a slope of 0 indicates no difference between shorter-term and longer-term memory processes, a negative slope indicates forgetting over longer delays, a positive slope indicates impaired shorter-term memory processes relative to longer-term memory processes).

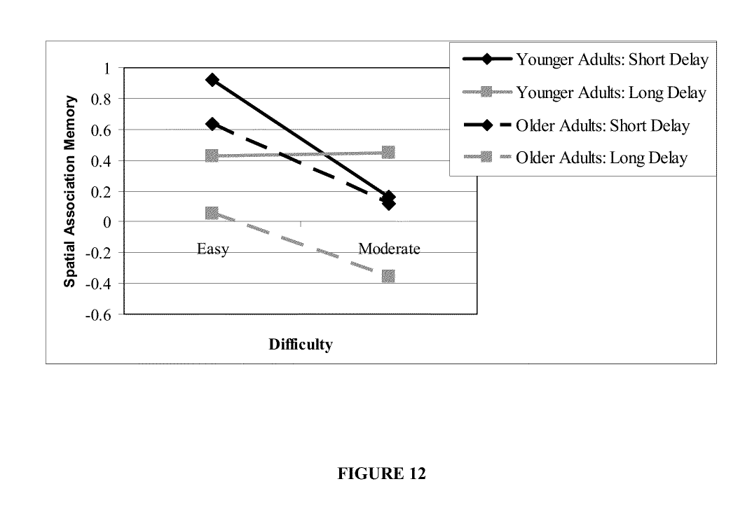

FIG. 12 is a graphical summary of data obtained from 37 younger adults and 16 older adults from the Spatial Association Memory task, in which the similarity of the presented images varied between the difficulty levels. The objects in the Moderate condition were visually more similar to each other (e.g., same color, similar shape) than the objects in the Easy Condition, and the delay between study and test images was either short (approximately 11 seconds) or long (approximately 45 seconds). Older adults show memory declines relative to younger adults at both short and long delays in the Easy condition. Performance declines for the younger adults in the Moderate condition, however, younger adults show higher levels of memory than the older adults at the long delay. The spatial association memory score used was (Duration of Viewing to the Critical Regions: Altered Image/Duration of Viewing to Critical Regions: First Presentation—Altered)−(Duration of Viewing to the Critical Regions: Repeated Image/Duration of Viewing to Critical Regions: First Presentation—Repeated).

### Example 4

### Task: Object-to-Object Associations

Rationale: examine visual memory for the relations among objects.

Procedure: Subjects are shown an image (e.g., real-world scene, object) for 3 sec.; subsequently an item (e.g face, object) is shown overlaid onto the image for 3 sec, thereby resulting in an associated pair. There are 18 pairs in total, and each pair is shown twice. In the test phase, a previously viewed image is presented for 1 sec, subsequently, 2 previously viewed items are presented overlaid onto the first image for 3 sec. One of the overlaid items is the ‘matching’ associated member for the background image and the other is a previously viewed, but nonmatching item. In the short delay condition, the test display is shown approximately 30 seconds after the initial presentation of the pair. In the long delay condition, the test display would occur approximately 3 minutes after the first presentation of the pair (intervening images will include two short delay). Presentation of the scenes is pseudo-randomized in order to accommodate the delay conditions. There is a 1-second fixation screen in between the presentation of each scene.

Duration of Task 5 minutes.

A score contrasting viewing to the match image versus viewing to the nonmatching image is generated for each eye movement measure. A score of 0 (or below) indicates that there is no memory that has been maintained for the associated pair (i.e., viewing of matching items is equal to viewing of nonmatching items). A score higher than 0 indicates that the subject has memory for the associated pairs. A slope can be generated for the ratios across the delays to determine the stability of memory over time (here, a slope of 0 indicates no change in memory over time, a negative slope indicates forgetting over longer delays, a positive slope indicates impaired shorter-term memory processes relative to longer-term memory processes).

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

We claim:

1. A method of assessing cognitive function in a subject, comprising the steps of:
(a) presenting a plurality of images to the subject, wherein the plurality of images comprises a first subset of images and a second subset of images;
(b) monitoring eye movements of the subject during presentation of the first subset of images to obtain first eye movement data;
(c) monitoring eye movements of the subject during presentation of the second subset of images to obtain second eye movement data;
(d) comparing the first eye movement data and the second eye movement data to determine an index of cognitive function; and
(e) correlating the index of cognitive function with a degree of cognitive function in the subject, thereby assessing the cognitive function,
wherein the monitoring steps are carried out using an optical eyetracking system.

2. The method of claim 1, wherein the cognitive function is memory.

3. The method of claim 1, wherein the cognitive function is an ability to process and distinguish emotions.

4. The method of claim 1, wherein the cognitive function is an ability to categorize objects.

5. The method of claim 1, wherein the cognitive function is visual attention.

6. The method of claim 1, wherein the cognitive function is comprehension of written or spoken language.

7. The method of claim 1, wherein the optical eyetracking system is an infrared eyetracking system.

8. The method of claim 1, wherein the eye movements monitored are selected from the group comprising spatial and temporal parameters related to eye fixations, saccades, smooth pursuit movements and pupil dilation.

9. The method of claim 1, wherein the eye movements monitored are selected from the group consisting of number of fixations, duration of fixations, number of regions fixated, location of fixations, spatial distribution of fixations, temporal order of fixations, saccades, constraint/entropy with the spatial and temporal distributions of fixations, comparison of the similarity of eye movement patterns across images, characteristics of eye fixations with respect to particular regions of interest in an image, the number of transitions between pre-specified regions of interest, and smooth pursuit movements.

10. The method of claim 1, wherein the first subset of images is a single image not previously viewed by the subject, and the second subset of images is a single image previously viewed by the subject.

11. The method of claim 1, wherein the first and second subset of images are presented simultaneously.

12. The method of claim 1, wherein the first and second subset of images are presented sequentially.

13. The method of claim 1, wherein the first subset of images comprises one or more images not previously viewed by the subject, and the second subset of images comprises one or more images previously viewed by the subject.

14. The method of claim 13, wherein the first and second subset of images are presented simultaneously.

15. The method of claim 13, wherein the first and second subset of images are presented sequentially.

16. The method of claim 1, wherein the first subset of images comprises first image, wherein the first image has not been previously viewed by the subject, and the second subset of images comprises a repeated presentation of the first image, and wherein the second eye movement data is obtained by monitoring the eye movements of the subject during each of the repeated presentations of the first image.

17. The method of claim 1, wherein the first subset of images and second subset of images each consist of images depicting a plurality of items in a defined spatial arrangement, wherein the first and second subsets differ only in the relative spatial arrangement of the plurality of items.

18. The method of claim 1, wherein the first subset of images includes images of faces expressing an emotion within an emotionally congruent context, and the second subset of images includes images of faces expressing an emotion within an emotionally incongruent context.

19. A system for assessing cognitive function in a subject, comprising:
a presentation module configured to present a first subset of images and a second subset of images to the subject;
an optical eyetracking module configured to monitor the eye movement of the subject during presentation of the first subset of images and second subset of images to generate first eye movement data and second eye movement data, respectively; and
a computing module communicatively linked to the optical eyetracking module and optionally the presentation module, wherein the computing module is configured to receive the first eye movement data and the second eye movement data, compare the first eye movement data and the second eye movement data to determine an index of cognitive function, and correlate the index of cognitive function with a degree of cognitive function in the subject, thereby assessing the cognitive function.

20. The system of claim 19, wherein the presentation module is a computing platform selected from the group consisting of a laptop, a home computer, and a tablet computing device.

21. The system of claim 19, wherein the optical eyetracking module is an infrared eyetracking device.

22. A computer program product for assessing cognitive function, the computer program product comprising code which, when loaded into memory and executed on a processor of a computing device, is adapted to carry out a method of assessing cognitive function in a subject, comprising the steps of:
(a) presenting a plurality of images to the subject, wherein the plurality of images comprises a first subset of images and a second subset of images;
(b) monitoring eye movement of the subject during presentation of the first subset of images to obtain first eye movement data;
(c) monitoring eye movement of the subject during presentation of the second subset of images to obtain second eye movement data;
(d) comparing the first eye movements data and the second eye movement data to determine an index of cognitive function; and
(e) correlating the index of cognitive function with a degree of cognitive function in the subject, thereby assessing the cognitive function.
